(19) United States
(12) Patent Application Publication
(10) Pub. No.: US 2021/0214711 A1
(43) Pub. Date: Jul. 15, 2021

(54) METHOD FOR OBTAINING FAT-SOLUBLE AND WATER-SOLUBLE COMPOUNDS FROM MICROALGAE BY MODULATING THE POLARITY OF VEGETABLE OR ANIMAL OILS

(71) Applicants: ALGAMA, EVRY (FR); UNIVERSITE D'AVIGNON ET DES PAYS DE VAUCLUSE, AVIGNON (FR)

(72) Inventors: Farid CHEMAT, MORIERES-LES-AVIGNON (FR); Maryline VIAN, ARAMON (FR); Lea VERNES, LE PONTET (FR); Thomas FELICE, CLAMART (FR)

(21) Appl. No.: 17/055,576

(22) PCT Filed: May 14, 2019
(86) PCT No.: PCT/FR2019/000074

§ 371 Date: Nov. 14, 2020

(30) Foreign Application Priority Data
May 14, 2018 (FR) FR1870559

(51) Int. Cl. C12N 13/00; B01D 11/02

(52) U.S. Cl. CPC C12N 13/00; B01D 11/0265

(57) ABSTRACT

The present invention relates to the field of converting algal biomass. The present invention relates to a method for obtaining fat-soluble and water-soluble compounds from a biomass of eukaryotic or prokaryotic microalgae (cyanobacteria), and the oil obtained by said method and the uses thereof, particularly in the food and food supplement sectors.

The present invention relates to the field of exploitation of algal biomass. The object of the present invention is a method for obtaining fat-soluble and water-soluble compounds from a biomass of eukaryotic or prokaryotic microalgae (cyanobacteria), and the oil obtained by said method and the uses thereof, in particular in the food sector or as food supplements.

Microalgae, cultivated for over 30 years, are a food product in themselves. Two approaches to exploitation exist in human and animal nutrition: the first consists in aiming to consume the entire microalgae, the second relates to the extraction, transformation and treatment of bioactive molecules originating therefrom. In France, just three species of microalgae are authorized for human consumption without processing, and have exceptional nutritional properties: spirulina or Arthrospira platensis, the green microalgae Chlorella, and the diatom Odontella aurita. These microorganisms are major elements in the issue of global food, owing to their unique protein content and the low environmental impact of their production, due to a low consumption of water and of energy.

Today consumers are turning increasingly to a healthier and more natural diet. They are looking for quality products to make up their everyday meals. In parallel with this mode of consumption, a growing number of people are turning to “alternative” medicines, in particular using traditional remedies in order to obtain the most natural possible treatment. However, a balanced diet is sometimes not sufficient for preventing deficiencies. Thus, food supplements have emerged, and have been largely democratized since the “plant” decree of 2014, permitting the use of several plants previously reserved for pharmaceuticals. Consumers now benefit from a large range of products for preventing various health problems, such as prevention of obesity, cardiovascular problems, improving concentration, etc. Omegas 3 and 6 are some of the popular molecules, due to the interest therein in health, and the large field of applications thereof. They are used in particular to prevent cardiovascular diseases. They are found in oily fish and seafood, but are also found, in non-negligible quantities, in microalgae. These fatty acids are considered essential since they are not man-made. They therefore have to be obtained through diet.

The mammalian organism is capable of synthesizing fatty acids from oleic acid (C18:1). However, this is not the case for some polyunsaturated fatty acids having 34 carbon atoms, which are synthesized only by bacteria or vegetables. Mammals can therefore obtain them only through food, which is why these fatty acids are considered essential. The two main fatty acids that are essential for humans are linoleic acid and α-linoleic acid. Other long-chain fatty acids, which are essential for the good functioning of the organism, are synthesized from these fatty acids. Thus, by adding a double bond and by lengthening the carbon chain, arachidonic acid is obtained from linoleic acid, and eicosapentaenoic acid (EPA) from α-linoleic acid. The fatty acids synthesized by means of said metabolism are used in various processes, such as signaling, inflammatory reactions, brain and retina development, etc. These are omega 3 (EPA, DHA).

The extraction of lipids, and in particular essential fatty acids, is frequently achieved in organic solvents originating from petrochemistry, such as hexane (Comparison of solvents for extraction of krill oil from krill meal: Lipid yield, phospholipids content, fatty acids composition and minor components. Xie et al, 2017). However, a major disadvantage of these organic solvents is that they are not suitable for consumption.

Manufacturers use fatty vegetable substances as a solvent in order to extract the fat-soluble active ingredients of the plant. They form the basis of several patents, such as patent FR3002845 B1, which protects a method for preparing a cosmetic and/or dermatological product based on a cork extract, at least one portion of the tree providing the cork, and at least one natural oily body, or patent FR2994840 B1, which protects an oily extract obtained by extraction by means of an oily excipient of non-volatile compounds contained in propolis. Other patents in a similar field have appeared, such as in particular patent FR2694300 B1 relating to a method for extracting and fixing aromatic compounds on a non-aqueous substrate.

However, none of these documents describes a flexible method allowing to extract both fat-soluble compounds and water-soluble compounds, in particular from a microalgae biomass, the product of which can be used directly for human or animal consumption.

The applicants have today developed a method which allows for industrial-scale obtention, from a starting microalgae biomass, of an oil enriched in all the microalgal fat-soluble and water-soluble compounds, i.e. not only the fat-soluble compounds, but also the water-soluble compounds. Indeed, due to the modulation of the extraction parameters, and the polarity of the oil, the method according to the invention makes it possible, furthermore, to obtain apolar compounds such as proteins, while monitoring the presence thereof. Furthermore, since the solvent used is a natural oil, without any residual trace of organic solvent, the extract is pure and suitable for food consumption as it is, and no evaporation step is required.

Thus, a first object of the present invention relates to a method for obtaining fat-soluble and water-soluble compounds from a biomass of eukaryotic or prokaryotic microalgae (cyanobacteria), characterized in that said method comprises

1. a step of mixing said biomass with oil, preferably vegetable, said oil comprising between 0.25% and 10% by mass of at least one amphiphilic additive, preferably being a monoglyceride, a diglyceride, or a phospholipid, allowing for modulation of the polarity of said oil, and

2. a step of macerating said biomass with said oil, allowing for the mixture to be homogenized, and/or
3. a step of extracting said fat-soluble and water-soluble compounds by means of ultrasound treatment with an application of ultrasonic power (Pus) of between 1 and 1000 W/L, applied to said biomass mixed with said oil obtained in step 1) at a temperature (T) of between 15 and 70° C., and/or
4. a step of extracting said fat-soluble and water-soluble compounds by means of treatment by electromagnetic microwaves with an application of power (W) of between 1 and 1000 W/L, applied to said biomass mixed with said oil obtained in step 1) at a temperature (T) of between 15 and 70° C.

For accuracy, the method according to the invention may comprise:

The mixing step 1) followed by the maceration step 2)
The mixing step 1) followed by an ultrasound treatment step 3)
The mixing step 1) followed by an electromagnetic microwave treatment step 4)
The mixing step 1) followed by a maceration step 2) and an ultrasound treatment step 3)
The mixing step 1) followed by a maceration step 2) and an electromagnetic microwave treatment step 4)
The mixing step 1) followed by an ultrasound treatment step 3) and an electromagnetic microwave treatment step 4)
The mixing step 1) followed by a maceration step 2), an ultrasound treatment step 3), and an electromagnetic microwave treatment step 4)

More preferably, according to the invention, the method according to the invention comprises:

The mixing step 1) followed by a maceration step 2) and an ultrasound treatment step 3), or
The mixing step 1) followed by a maceration step 2) and an electromagnetic microwave treatment step 4), or
The mixing step 1) followed by an ultrasound treatment step 3) and an electromagnetic microwave treatment step 4), or
The mixing step 1) followed by a maceration step 2), an ultrasound treatment step 3), and an electromagnetic microwave treatment step 4).

According to the present invention, fat-soluble compounds is intended to mean compounds that are soluble in a fatty substance and contained in microalgae and/or cyanobacteria, preferably selected from the short-chain saturated fatty acids, such as butyric acid, caproic acid, or caprylic acid, the long-chain saturated fatty acids such as capric acid, undecanoic acid, lauric acid, tridecanoic acid, myristic acid, pentadecanoic acid, palmitic acid, margaric acid, or stearic acid, very long-chain saturated fatty acids such as arachidic acid, docosanoic acid, tricosanoic acid, or tetracosanoic acid, monounsaturated fatty acids such as undecanoic acid, lauroleic acid, pentadecanoic acid, palmitoleic acid, heptadecanoic acid, oleic acid, eicosenoic acid, docosaenoic acid or tetracosenoic acid, polyunsaturated fatty acids such as hexadecadienoic acid, linoleic acid (precursor of omegas 3 and 6), alpha-linolenic acid (ALA, precursor of omegas 3 and 6), gamma-linolenic acid (GLA), eicosadienoic acid, dihomolinoleic acid, eicosapentaenoic acid (EPA), fat-soluble vitamins such as α-tocopherol (vitamin E), fat-soluble pigments such as chlorophyll, or carotenoids such as β-carotene (vitamin A precursor), lutein, asthaxanthin, zeaxanthin, cryptoxanthin (vitamin A precursor), lycopene, sterols, alkaloids, phenolic compounds such as flavonoids, or terpenes such as phytol (precursor of vitamin E).

According to the present invention, water-soluble compounds is intended to mean compounds that are soluble in water and are contained in microalgae and/or cyanobacteria, preferably selected from proteins and enzymes, the vitamins of groups B and C, minerals such as zinc, calcium, potassium, magnesium and iron, pigments such as phycocyanin, allophycocyanin or phycoerythrin, and essential amino acids.

According to the present invention, microalgae is intended to mean eukaryotic microalgae which are characterized by a cellular wall and a nucleus, comprising chlorophytes, chrysophytes and pyrrophytes, said eukaryotic microalgae typically being referred to as “microalgae,” and prokaryotic microalgae which do not have a nucleus or cellular wall, comprising cyanophytes, specifically referred to hereinafter as “cyanobacteria.”

According to the present invention, oil is intended to mean a fatty substance which is liquid at average temperature and insoluble in water, of vegetable, animal or mineral origin, and is compatible for food usage. Since said oil is substantially composed of triglyceride, it is completely apolar and initially makes it possible to extract only compounds of the same type as itself, i.e. very apolar compounds.

The oil used according to the invention is preferably a vegetable oil.

More preferably, the oil used according to the invention is selected from coconut oil, corn oil, cottonseed oil, olive oil, palm oil, groundnut oil, rapeseed oil, canola oil, safflower oil, sesame oil, soybean oil, sunflower oil, almond oil, beech nut oil, brasil nut oil, cashew nut oil, hazelnut oil, macadamia nut oil, mongongo nut oil, pecan nut oil, pine nut oil, pistachio oil, walnut oil, pumpkin seed oil, grapefruit seed oil, lemon oil, orange oil, bitter gourd oil, marrow oil, butternut squash seed oil, egusi seed oil, pumpkin seed oil, watermelon seed oil, acai oil, black seed oil, blackcurrant seed oil, borage seed oil, evening primrose oil, linseed oil, amaranth oil, apricot oil, apple seed oil, argan oil, avocado oil, babassu oil, ben oil, Borneo tallow nut oil, cape chestnut oil, carob pod oil (algaroba oil), cocoa butter oil, cocklebur oil, cohune oil, coriander oil, camelina oil, grapeseed oil, hemp oil, kapok seed oil, kenaf seed oil, lallemantia oil, mafura oil, marula oil, meadowfoam seed oil, mustard oil, niger seed oil, poppyseed oil, nutmeg oil, okra seed oil, papaya seed oil, perilla seed oil, persimmon seed oil, pequi oil, pili nut oil, pomegranate seed oil, poppy oil, pracaxi oil, virgin pracaxi oil, prune stone oil, quinoa oil, ramtil oil, rice bran oil, royal jelly, shea nut oil, sacha inchi oil, mamey oil, seje oil, shea butter oil, taramiara oil, tea seed oil, thistle oil, tigernut oil, tobacco seed oil, tomato seed oil, and wheat germ oil.

Even more preferably, the oil used according to the invention is sunflower oil.

An amphiphilic additive is intended to mean a compound having a fat-soluble part (long carbon chain) and a water-soluble part (ionic or not). These molecules have the particular characteristic of concentrating and collecting at the interfaces between water and other substances that are not easily soluble in water, such as fatty substances, in particular due to the free hydroxide groups thereof. Surfactants are examples of amphiphilic additives, and are classified according to the HLB (hydrophilic/lipophilic balance) index, the index 1 corresponding to oleic acid, and the index 20 corresponding to potassium oleate. This scale makes it possible to assess the rather fat-soluble or, in contrast, rather hydrophilic, nature of an amphiphile, and to identify the application for which it is best suited. A surfactant of an HLB<9 would be fat-soluble in character (in favor of water-in-oil emulsions), whereas a surfactant of an HLB>11 would be hydrophilic in character (in favor of oil-in-water emulsions). Using amphiphilic additive having different HLBs makes it possible to modulate the polarity of the oil used in the method according to the invention, and to extract less apolar compounds.

Preferably, the amphiphilic additive(s) according to the invention is/are selected from the monoglycerides such as monostearate (E471), sorbitan monooleate (Span 80, E494), sorbitan monolaurate (Tween 20, E432), polyoxyethylene sorbitan monopalmitate (Tween 40, E434), polyoxyethylene sorbitan monooleate (Tween 80 or polysorbate 80, E433), glyceryl monooleate (type 40, E491), mono- and diglycerides of fatty acids such as glycerol palmitostearate (E471), and the phosphatidylcholines, such as soya lectin (E322).

Modulation of the polarity of the oil means modifying the distribution of negative and positive charges of the oil, which, by reducing the apolar character of the oil, makes it possible to favor the extraction of polar (water-soluble) compounds, or, by increasing the apolar character, makes it possible to favor the extraction of apolar (fat-soluble) compounds.

Preferably, according to the invention, the biomass of eukaryotic or prokaryotic microalgae (cyanobacteria) is in dry, moist, or pre-extracted form.

Biomass in dry form is intended to mean, according to the invention, biomass that has undergone drying or lyophilization. Traditionally, biomass in dry form has a moisture content of approximately 10% water by weight, but the moisture content can be greater if the drying has not been carried out for as long. The drying step can be performed by any method known from the general teaching of a person skilled in the art, such as thermal drying, consisting for example in drying the biomass by means of the heat generated by natural gas combustion (conduction, convection, etc.), or even solar drying consisting in using the sun's rays in order to heat and dry the biomass, for example in outside pools. Examples of conditions for performing these drying steps can be found in the document “Les Différentes techniques de récolte de micro-algues: aspects techniques, économiques et environnementaux” [The various techniques for harvesting microalgae: technical, economic and environmental aspects], by Florian Delrue, published within the context of the Algo'Réso conference, Oct. 22, 2013, and available from https://www.mio.univ-amu.fr/IMG/pdf/20I3I022_02_presa_CEA.pdf

Biomass in moist form is intended to mean, according to the invention, biomass that has not undergone a drying step, and thus has a high moisture level, in the region of 75% water by weight.

Biomass in pre-extracted form is intended to mean, according to the invention, biomass that has undergone preliminary extraction, in particular water-soluble compounds, for example a mechanical extraction step. This pre-extracted biomass can be in the form of a lyophilized pellet.

Preferably, according to the invention, in the case where the method comprises a step 3) of ultrasound treatment, the ultrasound power and the temperature are modulated in order to vary the type and the quantity of compounds extracted from said biomass.

Ultrasound treatment with application of ultrasound power (Pus) is intended to mean, according to the present invention, physical treatment that makes use of an ultrasound reactor. Preferably, according to the invention, the ultrasound power is between 1 and 1000 W/L, applied to the microalgae or to the cyanobacteria mixed with the oil and said at least one amphiphilic additive, preferably between 30 and 100 W/L, more preferably between 50 and 80 W/L. Preferably, the energy employed by the ultrasound reactor varies between 500 and 5000 J, the feed output of the ultrasound reactor varies from 1 L/hour to 1000 L/hour, preferably 1 L/hour, and the ultrasound treatment is carried out for a duration that varies between 30 seconds and 1 hour.

Preferably, according to the invention, in the case where the method comprises a step 4) of electromagnetic microwave treatment, the power, the exposure number and the temperature are modulated in order to vary the type and the quantity of compounds extracted from said biomass.

Preferably, according to the invention, the modulation of the power of the electromagnetic microwaves makes it possible to increase or reduce the quantity of fat-soluble and water-soluble compounds extracted from said biomass, each of said compounds having an optimal power where a maximum thereof is extracted, for example 850 W for chlorophyll a and 1000 W for the carotenoids. Using other powers for said compounds thus makes it possible to reduce the quantity thereof that is extracted.

Treatment by electromagnetic microwaves is intended to mean, according to the present invention, treatment of the biomass using electromagnetic waves that are imposed by a microwave generator.

Exposure number is intended to mean the number of repetitions of the microwave treatment.

Preferably, according to the invention, the treatment takes the form of at least one exposure of from 30 seconds to 30 minutes. Even more preferably, according to the invention, the treatment takes the form of a plurality of exposures each lasting from 30 seconds to 30 minutes.

Preferably, according to the invention, the form of the original biomass, the type and quantity of the oil and of said at least one amphiphilic additive, and step 3) of ultrasound treatment and/or 4) of electromagnetic microwave treatment are modulated in order to vary the type and the quantity of the fat-soluble and water-soluble compounds extracted from said eukaryotic microalgae or cyanobacteria.

The modulation of the form of the original biomass makes it possible to increase or reduce the quantity of fat-soluble and water-soluble compounds extracted from said biomass. Using pre-extracted biomass makes it possible to extract more compounds than in the case of a dry biomass, and using said dry biomass makes it possible to extract more compounds than in the case of a moist biomass.

Modulation by the type and the quantity of oil makes it possible to

Modulation by the type and the quantity of amphiphilic additive affects the yields for extraction of fat-soluble and water-soluble compounds, whatever the technique used. Adding for example Tween 80 or Span 80 makes it possible to increase the quantity of compounds extracted, in the case of ultrasound treatment or in the case of microwave treatment. Treatment by maceration has also made it possible to identify that adding Span 80 makes it possible to increase the yield of extraction of carotenoids and of chlorophyll a, whereas adding Tween 80 makes it possible to increase only the yield of extraction of chlorophyll a, the yield of extraction of carotenoids reducing.

The modulation of step 3) of ultrasound treatment makes it possible to increase or reduce the quantity of fat-soluble and water-soluble compounds extracted from said biomass, each of said compounds having an optimal ultrasound power where a maximum thereof is extracted.

The modulation of step 4) of electromagnetic microwave treatment makes it possible to increase or reduce the quantity of fat-soluble and water-soluble compounds extracted from said biomass, each of said compounds having an optimal power where a maximum thereof is extracted, for example 850 W for chlorophyll a and 1000 W for the carotenoids. Using other powers for said compounds instead of said optimal power will thus result in a reduction of the quantity extracted.

Preferably, according to the invention, the duration of step 3) and/or of step 4) is in each case between 30 seconds and 1 hour.

Even more preferably, according to the invention, the duration of step 3) and of step 4) is in each case between 30 seconds and 30 minutes.

Preferably, according to the invention, the fat-soluble compounds are selected from the short-chain saturated fatty acids, such as butyric acid, caproic acid, or caprylic acid, the long-chain saturated fatty acids such as capric acid, undecanoic acid, lauric acid, tridecanoic acid, myristic acid, pentadecanoic acid, palmitic acid, margaric acid, or stearic acid, very long-chain saturated fatty acids such as arachidic acid, docosanoic acid, tricosanoic acid, or tetracosanoic acid, monounsaturated fatty acids such as undecanoic acid, lauroleic acid, pentadecanoic acid, palmitoleic acid, heptadecanoic acid, oleic acid, eicosenoic acid, docosaenoic acid or tetracosenoic acid, polyunsaturated fatty acids such as hexadecadienoic acid, linoleic acid (precursor of omegas 3 and 6), alpha-linolenic acid (ALA, precursor of omegas 3 and 6), gamma-linolenic acid (GLA), eicosadienoic acid, dihomolinoleic acid, eicosapentaenoic acid (EPA), fat-soluble vitamins such as α-tocopherol (vitamin E), or fat-soluble pigments such as chlorophyll, or carotenoids such as β-carotene (vitamin A precursor), lutein, asthaxanthin, zeaxanthin, cryptoxanthin (vitamin A precursor), or lycopene.

Preferably, according to the invention, the water-soluble compounds are selected from proteins and enzymes, the vitamins of groups B and C, minerals such as zinc, calcium, potassium, magnesium and iron, pigments such as phycocyanin, allophycocyanin or phycoerythrin, essential amino acids, polysaccharides, and fibers.

Preferably, according to the invention, the eukaryotic microalgae are selected from Chlorella, Nannocloropsis, Duanaliella and Euglena, and the cyanobacteria are selected from spirulina (Arthrospira platensis or Spirulina maxima) and AFA (Aphanizomenon Flos-aquae).

Preferably, according to the invention, the method comprises an additional step 5) of solid/liquid separation, preferably by means of centrifugation, of the extract obtained after step 2), 3) or 4).

Preferably, according to the invention, the method comprises an additional step 6) of filtration of the liquid part obtained by separation of the extract obtained after step 5).

Preferably, according to the invention, the microalgae or cyanobacteria/oil ratio is between ⅕^{th} and 1/50^{th} of the volume, preferably 1/20^{th} of the volume.

According to a second aspect, the invention relates to an oil enriched in fat-soluble and water-soluble compounds of eukaryotic or prokaryotic microalgae (cyanobacteria), such as obtained by the method according to the invention.

Oil enriched in fat-soluble and water-soluble compounds of eukaryotic or prokaryotic microalgae (cyanobacteria) is intended to mean an oil comprising water-soluble and fat-soluble compounds that are initially not present in the oil, and/or an increase in the concentration of water-soluble and fat-soluble compounds initially present in the oil in an extremely low quantity (amount less than a microgram).

Preferably, the invention relates to an oil enriched in water-soluble and fat-soluble compounds that are initially not present in the oil.

More particularly, said method makes it possible to extract the fat-soluble compounds and the water-soluble compounds from the microalgae and cyanobacteria. Preferably, the extract produced will have organoleptic qualities, as well as a nutritional composition that is optimal for human consumption.

According to a third aspect, the invention relates to the use of according to the invention in chemical, food, cosmetic or pharmaceutical, preferably food, compositions.

Given the advantages, set out above, which said filtrates/extracts exhibit, the applications thereof in the field of diet, medicine or cosmetics, in particular as food supplements, are of particular interest. The invention also targets, as novel products, the concentrates obtained at the end of the filtration step. These concentrates can also be used in the field of diet, medicine or cosmetics, or as food supplements.

### DESCRIPTION OF THE FIGURES

FIG. 1 Shows absorption spectra of the oil alone (A), the oil enriched with spirulina compounds (B) after the biomass of Arthrospira platensis has been mixed into the extraction solvent (sunflower vegetable oil at 1% Tween 80) according to the ratio 1/20 (0.5 g of spirulina in 10 g of oil), and the mixture has been introduced into a stirred ball mill having ceramic balls and subjected to rotations of a speed of 4000 rotations/minute for 1 hour at ambient temperature, and then centrifuged for 10 minutes at 9000 rpm.

FIG. 2 Shows a quantitative analysis carried out by means of spectrophotometric measurements in order to evaluate the chlorophyll a and carotenoid content depending on the biomass type (moist, dry or pre-extracted) and the amphiphilic additive type (none, Tween 80, or Span 80), after having introduced the mixture into a stirred ball mill for 1 hour at 4000 rpm at ambient temperature. The first line shows a control value. The following 3 lines show the data after using moist biomass. The following 3 lines show the data after using dry biomass. The following 3 lines show the data after using pre-extracted biomass, in this case a lyophilized pellet.

FIG. 3: Shows a quantitative analysis of neutral lipids, carried out by means of high-performance thin-layer chromatography (HPTLC). 20 μL of the sample, at 1 mg/mL, were deposited in chloroform. The presence of monoglycerides (MAG), free fatty acids (FFA), triglycerides (TAG), diglycerides (DAG) and sterols (STE) is studied.

FIG. 4: Shows a quantitative analysis carried out by means of spectrophotometric measurements in order to evaluate the chlorophyll a and carotenoid content depending on the extraction technique (stirred ball mill, maceration, microwaves or ultrasound) and the amphiphilic additive type (none, Tween 80, or Span 80), using a moist biomass (80% moisture). The first line shows a control value. The following 3 lines show the data after maceration for 24 hours. The following 3 lines show the data after treatment with electromagnetic microwaves, 4 times a minute. The following 3 lines show the data after ultrasound treatment with application of ultrasonic power for 30 minutes. The last 3 lines show the data after treatment using a stirred ball mill for 1 hour.

FIG. 5: Shows a schematic diagram of the microwave application cycles. The rising curves represent the points of microwave treatment.

FIG. 6: Shows photographs which characterize the extractions achieved by means of microwave treatment at powers of 300, 600, 850 and 1000 W, and according to the amphiphilic additive type (none, Tween 80, or Span 80).

FIG. 7: Shows a quantitative analysis carried out by means of spectrophotometric measurements in order to evaluate the chlorophyll a and carotenoid content depending on the power of the microwave treatment (300, 600, 850 and 1000 W). The first 3 lines show the data in the case of treatment at 1000 W. The following 3 lines show the data in the case of treatment at 850 W. The 3 lines show the data in the case of treatment at 600 W. The last 3 lines show the data in the case of treatment at 300 W.

### EXAMPLES

### Example 1

### Study of the Modularity of the State of the Biomass and of the Addition of Amphiphilic Additive by Enrichment of Sunflower Oil Using a Stirred Ball Mill

Moist, dry or pre-extracted biomass (Arthrospira platensis) is mixed with the extraction solvent (sunflower vegetable oi, sunflower vegetable oil at 1% Tween 80, and sunflower vegetable oil at 1% Span 80) according to the ratio 1/20; i.e. 1 g of biomass for 20 g of solvent. In this case, 0.5 g of spirulina is introduced into 10 g of oil. The mixtures are then introduced into a stirred ball mill having ceramic balls, and subjected to rotations of a speed of 4000 rotations/minute for 1 hour, at ambient temperature. The extracts are then centrifuged for 10 minutes at 9000 rpm in order to achieve a clear oil.

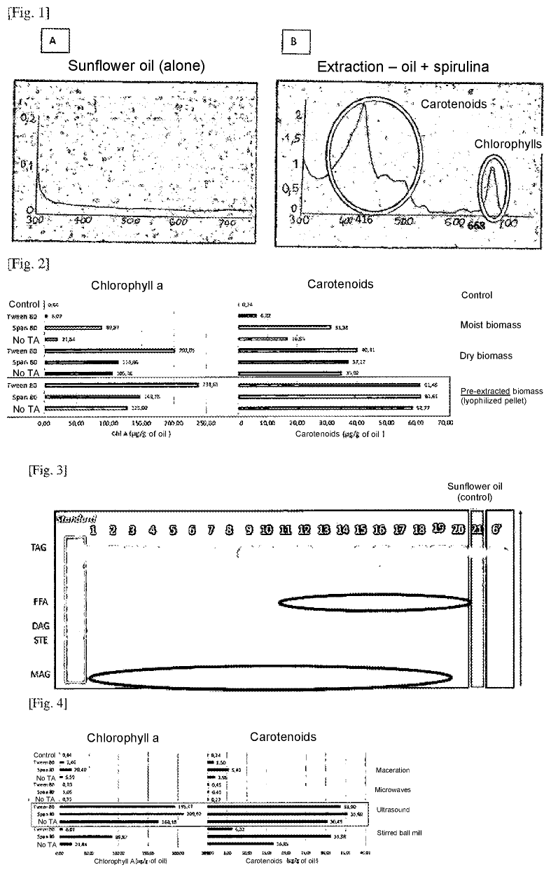

The oil collected after these extractions is of a green color which is different from the initial color of the sunflower oil at the outset (light yellow). Comparing the absorption spectra of the oil alone (FIG. 1A) and the sunflower vegetable oil at 1% Tween 80 enriched in spirulina compounds (FIG. 1B) reveals the presence of peaks characteristic of vegetable pigments: carotenoids (λₘₐₓ=416 nm) and chlorophyll (λₘₐₓ=668 nm). Thus, according to these preliminary qualitative observations, the sunflower oil appears to have been enriched with spirulina pigments by the method according to the invention.

A quantitative analysis was then carried out by means of spectrophotometric measurements in order to evaluate the content of each of the pigments thereof, depending on the biomass and amphiphilic additive type. The results obtained (FIG. 2) demonstrate that the biomass type modulates the pigment content. Indeed, the maximum pigment contents are achieved in the case of a pre-extracted biomass (lyophilized pellet, having already undergone extraction). Modulating the polarity by adding amphiphilic additive into the oil also affects the pigment yields. Thus, in the case of the surfactant Tween 80, the extract contains 238.61 μg of chlorophyll a per g of oil, and 61.46 μg of carotenoids per g of oil.

Furthermore, focusing on the lipids classes, the enrichment of vegetable oil with neutral lipids of spirulina following extraction is also evident. A qualitative analysis of the neutral lipids has been carried out (FIG. 3) by means of high-performance thin-layer chromatography (HPTLC), where 20 μL of the sample, at 1 mg/mL, was deposited in chloroform. The results obtained show the presence of monoglycerides (MAG) in extracts 1 to 19 but not in the control, and the presence of free fatty acids (FFA) in extracts 12 to 19 but not in the control. These results highlight the presence of neutral lipids such as free fatty acids (FFA) and monoglycerides (MAG) in vegetable oil following extraction. The vegetable oil, which did not contain these compounds in the initial state, has therefore been enriched.

### Example 2

### Study of the Modularity of the Extraction Technique—Enrichment of Sunflower Oil Using Different Extraction Methods

For a given biomass (in this case moist biomass (80% moisture) of Arthrospira platensis), different extraction methods have been used in order to enrich the sunflower oil:

Stirred ball mill: 4000 rpm for 1 hour, using 20 g of ceramic balls
Ultrasound: 25 kHz, 150 W, for 30 min
Microwaves: test using different powers of from 300 to 1000 W, by means of treatment by 1-minute cycles

### Maceration.

The spectrophotometric measurements carried out (FIG. 4) show that the yields of chlorophyll a and carotenoids are modulated by the extraction technique selected. Indeed, the maximum yields are achieved in the case of ultrasound extraction: 209.92 μg of chlorophyll a per g of oil, and 35.60 μg of carotenoids per g of oil (in the case of oil at 1% Span 80). In this case, adding Span 80 to the oil modulates the polarity in favor of the extraction of chlorophyll and carotenoid pigments.

### Example 3

### Study of the Modularity of the Extraction Parameters—Enrichment of Sunflower Oil Using Microwaves

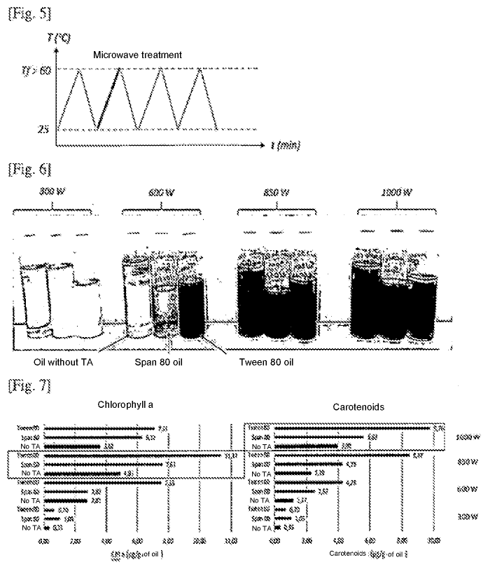

In order to highlight the modularity of the extraction technique, various parameters have been tested. The biomass selected for these extractions is a spirulina paste having 79.87% moisture. The biomass/oil mixture is made at a dry ratio of 1/20^{th}. A plurality of cycles of exposure to microwaves, of a duration of 1 minute, were applied to the solvent/biomass mixture, interspersed with cooling in an ice bath (shown in FIG. 5).

Extractions have been carried out by microwave treatment at powers of 300, 600, 850 and 1000 W. FIG. 6 shows that the oil becomes darker and darker as the microwave power increases, showing that the pigment extraction is thus favored by an increased microwave power. Furthermore, modulating the polarity also plays a significant role in the extraction of pigments, since it is possible to identify a color difference depending on the amphiphilic additive used, for the same power. Thus, at 600 W, oil comprising 1% Tween 80 has a more intense color than the extracts achieved using oil alone and using oil with added Span 80, at 1%.

The results shown in FIG. 7 confirm the observations of the above paragraph. The greater the microwave power, the greater the pigment yield. In the same way, adding Tween 80 to the oil allows for a greater yield than that achieved using oil alone, or using oil with Span 80 added: 11.37 μg of chlorophyll a per g of oil, and 9.76 μg of carotenoids per g of oil in the case of oil with Tween 80 added at 1%, at 850 and 1000 W, respectively.

### Conclusions

The results obtained in these three examples demonstrate that the modularity of the biomass type affects the quantity of fat-soluble and water-soluble compounds extracted. Indeed, using a pre-extracted biomass will allow for better extraction of fat-soluble and water-soluble compounds than in the case of a dry biomass, although said dry biomass makes it possible to achieve better extraction of fat-soluble and water-soluble compounds than in the case of extraction from moist biomass.

The results obtained in these three examples also demonstrate that the extraction technique used modulates the yields for extraction of fat-soluble and water-soluble compounds. Indeed, using ultrasound makes it possible to achieve greater yields of carotenoid and chlorophyll a extraction, compared with maceration, microwave treatment, or using a stirred ball mill.

The results obtained in these three examples also demonstrate that the modulation of the technique based on microwaves influences the yields for extraction of fat-soluble and water-soluble compounds. Indeed, a maximum quantity of carotenoids is extracted at the microwave power of 1000 W, whereas a maximum of chlorophyll a is extracted at 850 W.

Indeed, the results obtained in these three examples demonstrate that adding amphiphilic additive affects the yields for extraction of fat-soluble and water-soluble compounds, whatever the technique used. Adding for example Tween 80 or Span 80 makes it possible to increase the quantity of compounds extracted, in the case of ultrasound treatment or in the case of microwave treatment. Treatment by maceration has also made it possible to identify that adding Span 80 makes it possible to increase the yield of extraction of carotenoids and of chlorophyll a, whereas adding Tween 80 makes it possible to increase only the yield of extraction of chlorophyll a, the yield of extraction of carotenoids reducing.

1. Method for obtaining fat-soluble and water-soluble compounds from a biomass of eukaryotic or prokaryotic microalgae (cyanobacteria), characterized in that said method comprises:
1. a step of mixing said biomass with oil, preferably vegetable, said oil comprising between 0.25% and 10% by mass of at least one amphiphilic additive, preferably being a monoglyceride, a diglyceride, or a phospholipid, allowing for modulation of the polarity of said oil, and
2. a step of macerating said biomass with said oil, allowing for the mixture to be homogenized, and/or
3. a step of extracting said fat-soluble and water-soluble compounds by means of ultrasound treatment with an application of ultrasonic power (Pus) of between 1 and 1000 W/L, applied to said biomass mixed with said oil obtained in step 1) at a temperature (T) of between 15 and 70° C., and/or
4. a step of extracting said fat-soluble and water-soluble compounds by means of treatment by electromagnetic microwaves with an application of power (W) of between 1 and 1000 W/L, applied to said biomass mixed with said oil obtained in step 1) at a temperature (T) of between 15 and 70° C.

2. Method according to the preceding claim, characterized in that the biomass of eukaryotic or prokaryotic microalgae (cyanobacteria) is in dry, moist, or pre-extracted form.

3. Method according to either of the preceding claims, characterized in that the duration of step 3) and/or of step 4) is in each case between 30 seconds and 1 hour.

4. Method according to any of the preceding claims, characterized in that the fat-soluble compounds are selected from the short-chain saturated fatty acids, such as butyric acid, caproic acid, or caprylic acid, the long-chain saturated fatty acids such as capric acid, undecanoic acid, lauric acid, tridecanoic acid, myristic acid, pentadecanoic acid, palmitic acid, margaric acid, or stearic acid, very long-chain saturated fatty acids such as arachidic acid, docosanoic acid, tricosanoic acid, or tetracosanoic acid, monounsaturated fatty acids such as undecanoic acid, lauroleic acid, pentadecanoic acid, palmitoleic acid, heptadecanoic acid, oleic acid, eicosenoic acid, docosaenoic acid or tetracosenoic acid, polyunsaturated fatty acids such as hexadecadienoic acid, linoleic acid (precursor of omegas 3 and 6), alpha-linolenic acid (ALA, precursor of omegas 3 and 6), gamma-linolenic acid (GLA), eicosadienoic acid, dihomolinoleic acid, eicosapentaenoic acid (EPA), fat-soluble vitamins such as α-tocopherol (vitamin E), or the fat-soluble pigments such as chlorophyll, or carotenoids such as β-carotene (vitamin A precursor), lutein, asthaxanthin, zeaxanthin, cryptoxanthin (vitamin A precursor), lycopene, sterols, alkaloids, phenolic compounds such as flavonoids, or terpenes such as phytol (precursor of vitamin E).

5. Method according to any of the preceding claims, characterized in that the eukaryotic microalgae are selected from Chlorella, Nannocloropsis, Duanaliella and Euglena, and in that the cyanobacteria are selected from spirulina (Arthrospira platensis or Spirulina maxima) and AFA (Aphanizomenon Flos-aquae).

6. Method according to any of the preceding claims, characterized in that it comprises an additional step 5) of solid/liquid separation, preferably by means of centrifugation, of the extract obtained after step 2), 3) or 4).

7. Method according to the preceding claim, characterized in that it comprises an additional step 6) of filtering the liquid part obtained by separation of the extract obtained after step 5).

8. Method according to any of the preceding claims, characterized in that the microalgae or cyanobacteria/oil ratio is between ⅕^{th} and 1/50^{th} of the volume, preferably 1/20^{th} of the volume.

9. Use of the method according to any of the preceding claims, where, in the case of the method comprising a step 3) of ultrasound treatment, the ultrasound power and the temperature are modulated in order to vary the type and the quantity of compounds extracted from said biomass.

10. Use of the method according to claims 1 to 8, where, in the case of the method comprising a step 4) of electromagnetic microwave treatment, the power, the exposure number and the temperature are modulated in order to vary the type and the quantity of compounds extracted from said biomass.

11. Use of the method according to claims 1 to 8, where the form of the original biomass, the type and quantity of the oil, and the type and quantity of said at least one amphiphilic additive, and the step 3) of ultrasound treatment and/or 4) of electromagnetic microwave treatment are modulated in order to vary the fat-soluble and water-soluble compounds extracted from said eukaryotic microalgae or cyanobacteria.

12. Oil enriched in fat-soluble and water-soluble compounds of eukaryotic or prokaryotic microalgae (cyanobacteria), such as obtained by the method according to claims 1 to 8.

13. Use of the enriched oil according to the preceding claim in chemical, food, cosmetic or pharmaceutical, preferably food, compositions.
